(19) United States
(12) Patent Application Publication
(10) Pub. No.: US 2007/0093410 A1
(43) Pub. Date: Apr. 26, 2007

(54) Selective inhibition of proteasomes of tuberculosis and other bacteria

(75) Inventor: Alfred L. Goldberg, Chestnut Hill, MA (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 11/510,125

(22) Filed: Aug. 25, 2006

Related U.S. Application Data
(63) Continuation of application No. PCT/US05/06270, filed on Feb. 25, 2005.
(60) Provisional application No. 60/547,813, filed on Feb. 26, 2004.

(51) Int. Cl. A61K 38/16; A61K 38/05; A61K 38/04

(52) U.S. Cl. 514/2; 514/18; 514/19; 514/64

(57) ABSTRACT

Compositions and methods for inhibiting bacterial proteasomes are provided. Methods of screening antibacterial compounds, methods of treating bacterial infections and disorders associated with bacterial infections, and methods of treating polyglutamine disorders are also provided.

### RELATED APPLICATIONS

This application is a continuation of PCT application no. PCT/2005/006270, designating the United States and filed Feb. 25, 2005; which claims the benefit of the filing date of U.S. Provisional Patent Application No. 60/547,813 filed on Feb. 26, 2004; both of which are hereby incorporated herein by reference in their entirety for all purposes.

### STATEMENT OF GOVERNMENT INTERESTS

This invention was made with Government support under Grant Number GM 46147-10 awarded by the National Institutes of Health. The Government has certain rights in the invention.

### FIELD OF THE INVENTION

The present invention relates to novel antimicrobial compounds that selectively inhibit proteasomes of bacteria while having minimal or no effect on mammalian proteasomes, pharmaceutical compositions of the antimicrobial compounds, methods of screening antimicrobial compounds and methods of treating bacterial infections and disorders associated with bacterial infections, and methods of treating polyglutamine disorders.

### BACKGROUND OF THE INVENTION

The major site of degradation of proteins in mammalian cells is the 26S proteasome complex. It is composed of the cylindrical 20S proteasome which degrades proteins to small peptides, surrounded by one or two 19S regulatory complexes which bind the protein substrate and then unfold and translocate them into the 20S proteasome for destruction (see Goldberg et al. (2001) Scientific American Jan: 68; Voges et al. (1999) Ann. Rev. Biochem. 68:1015; Coux et al. (1996) Ann. Rev. Biochem. 65:801). In eukaryotic cells, most proteins destroyed by the proteasome are first targeted for degradation by covalent linkage to multiple ubiquitin molecules in eukaryotic cells (Glickman et al. (2002) Physiol. Rev. 82:373). This process serves many essential roles in the cell, including selective degradation of unfolded proteins (Goldberg (2003) Nature 426:895), and regulation of many key cellular processes, including cell cycle, gene transcription, and apoptosis. One mammalian proteasome inhibitor, the dipeptide boronate VELCADE™ (i.e., bortezomib, available from Millennium Pharmaceuticals, Inc., Cambridge, Mass.), has recently been approved for treatment of the human cancer, multiple myeloma, and presently is in clinical trials for a variety of other cancers.

The eukaryotic proteasome contains six active sites: two are chymotrypsin-like in specificity and cleave after hydrophobic residues, two are trypsin-like and cleave after basic residues, and two are caspase-like and cleave after acidic groups in proteins (Kisselev et al. (2001) Chemistry and Biology 8:739). Most of the known proteasome inhibitors are dipeptide or tripeptide derivatives that bind to the chymotrypsin-like site and therefore comprise hydrophobic amino acids linked to an inhibitor group.

All proteasomes hydrolyze peptide bonds by a unique catalytic mechanism that distinguishes them from the other main families of proteolytic enzymes (e.g., the serine, cysteine, acidic or metallo-proteases). In the proteasomes of both eukaryotes and prokaryotes, the nucleophilic attack on the peptide bond occurs through the threonine residue on the N-terminus of the 20S proteasome's μ-subunits. Because of this unique mechanism, it has been possible to develop several types of pharmacological inhibitors that inhibit the proteasome selectively without affecting native proteases in the organism.

Prokaryotic proteasomes have a simpler subunit composition than eukaryotic proteasomes. All 20S proteasomes are composed of 4 superimposed rings, each of which contains 7 subunits. In most prokaryotes (e.g. mycobacteria, archaebacteria), there is one type of α subunit in the two outer rings and one type of β subunit in the inner rings. Only certain types of bacteria and archaebacteria contain proteasomes (for a review, see Zwickl, Goldberg, and Baumeister In: Wolf, D H, and Hilt, editors. Proteasomes: The World of Regulatory Proteolysis. Georgetown, Tex.: R.G. Landes Bioscience Publishing Co.; 2000. p. 8-20), but among them are mycobacteria, including the highly pathogenic Mycobacterium tuberculosis, and the well-characterized proteasomes of the archaebacterium, Thermoplasma acidophilum, whose structure and mechanism have been extensively studied (see Voges et al. (1999) Ann. Rev. Biochem. 68:1015).

Prokaryotes do not contain ubiquitin or 19S regulatory complexes, but do contain a 20S proteasome particle, which functions together in protein degradation with an ATPase ring (e.g., the AIDS-related complex (ARC) in eubacteria and proteasome-activating nucleotidase (PAN) in archaebacteria), which is homologous to the ring of ATPases at the base of the eukaryotic 19S complex. Based on studies of the PAN ATPase complex in archaebacteria, it is clear that these ATPases all bind substrates and translocate them into the archaebacterial 20S proteasome for degradation (Zwickl et al. (1999) J Biol. Chem. 274:26008; Navon et al. (2001) Mol. Cell. 8:1339; Benaroudj et al. (2003) Mol. Cell 11:69). In these prokaryotes, as in eukaryotes (e.g., mammals), the 20S particle is a 4-ring cylindrical structure within which proteins are digested to small peptides. However, the bacterial proteasomes only contain one type of active site, of which one is located on each of the seven β subunits in its central two rings. In contrast to the three types of active sites of narrow specificity of eukaryotic (e.g., mammalian) proteasomes, these various active sites are identical and of broad specificity and can cleave multiple types of bonds in proteins (set forth below).

There are a variety of known proteasome inhibitors (for a review, see Kisselev and Goldberg (2001) supra; Lee and Goldberg (1998) Trends in Cell Biol. 8:397). All bind to the active sites in the 20S particle. For example, peptide aldehydes (e.g. MG 132 or PSI) are widely used competitive inhibitors whose aldehyde group forms a complex (resembling the enzyme's transition state) with the proteasome's active site threonine. A very potent class of competitive inhibitors is the peptide boronate class, in which an active “warhead” on the peptide is a boronate group that also forms a transition-state complex with the active site threonine (Adams et al. (1999) Cancer Res. 59:2615). One irreversible class is composed of tripeptides with a vinyl sulfone group (in place of the aldehyde group); the vinyl sulfone covalently reacts with the catalytic threonine residue in the active site. Agents of the epoximicin family are peptide epoxyketones, originally of microbial origin. They also covalently modify the active site threonines on the different active sites. In all these cases, the specificity is determined by the nature of the attached peptide, which determines which active site the inhibitor binds to. A final class of proteasome inhibitors is the lactacystin homologs, the active forms of which are β-lactones, which form a covalent adduct with the active site threonines (see Kisselev and Goldberg (2001) supra).

Because it is highly desirable for therapy of bacterial disorders to find new types of antibiotics active against pathogenic mycobacteria (many of which are now resistant to standard antibiotic regimens), it is important to find an agent that inhibits or inactivates the mycobacterium without affecting host proteasomes and thus make them most sensitive to killing by the host macrophages. The proteasome inhibitors now available, though useful against life-threatening conditions such as cancers or stroke (due to the strong anti-inflammatory actions of proteasomes (see Goldberg and Rock (2002) Nat. Med 8:338)), can induce apoptosis (programmed cell death) in normal cells. Consequently, although proteasome inhibitors should, in theory, be useful in a number of disease states (e.g., tuberculosis), these agents are dangerous even at modest concentrations and have a limited therapeutic window due to their potential toxicity to the host. Accordingly, using proteasome inhibitors as antibacterial agents requires the design of proteasome inhibitors specific to the bacterial proteasomes without affecting the activity of mammalian proteasomes.

### SUMMARY OF THE INVENTION

The present invention is based in part on the discovery of a sequence that is only degraded by archaebacterial proteasomes (Venkatraman et al. (2004) Mol. Cell 14:95, incorporated herein by reference in its entirety for all purposes, and data set forth herein). The active sites of the 20S proteasome from archaebacteria are capable of rapidly cleaving sequences that mammalian proteasomes cannot cleave. Surprisingly, it has been discovered that a blocked tripeptide X-glutamine-glutamine-glutamine (xQQQ) sequence can selectively bind active sites of prokaryotic proteasomes without binding the chymotrypsin-like, trypsin-like or caspase-like active sites of eukaryotic proteasomes.

The present invention provides new antibiotic compounds useful for treating bacterial infections such as, for example, infections caused by the bacterium Mycobacterium tuberculosis. The present invention is directed in part to compounds (e.g., peptides) that inhibit bacterial proteasome activity while minimally affecting the activity of mammalian proteasomes.

Certain embodiments of the present invention are directed to methods for therapeutically treating a bacterial infection and methods for treating one or more symptoms associated with a bacterial infection in a human or non-human mammal in need thereof. In certain aspects, the bacterial infection is a Mycobacterium tuberculosis infection. The methods include administering to the human or non-human mammal a glutamine-glutamine dipeptide, a glutamine-glutamine-glutamine tripeptide or a polypeptide comprising a polyQ domain, and a pharmaceutically acceptable carrier. In certain aspects, the dipeptide, tripeptide or polypeptide includes an amino-terminal blocking moiety, such as N-acetyl, N-formyl, tert-butylcarbonyl, para-nitrophenylfonate and the like. In other aspects, the dipeptide, tripeptide or polypeptide includes a carboxy-terminal group that reacts with an active site of a proteasome, such as boronic acid, aldehyde, vinyl sulfone, epoxyketone, a beta lactone ring and the like. In certain aspects, the methods provided herein inhibit an activity of a bacterial proteasome. Certain aspects of the present invention are directed to the use of xQQQ-aldehyde, xQQQ-boronate, xQQQ-epoxyketone, xQQ-aldehyde, xQQ-boronate and/or xQQ-epoxyketone peptides to selectively inactivate microbial proteasomes. In certain aspects, symptoms associated with a bacterial infection include chest pain, non-productive coughing, coughing up blood, coughing up sputum, weakness, fatigue, weight loss, loss of appetite, chills, fever and night sweats.

Other embodiments of the present invention are directed to methods for killing a cell infected with a bacterium or for killing a bacterial cell. The methods include contacting the cell (i.e., the infected cell and/or the bacterial cell) with a glutamine-glutamine dipeptide, a glutamine-glutamine-glutamine tripeptide or a polypeptide comprising a polyQ domain, inhibiting an activity of a bacterial proteasome, and allowing the cell infected with a bacterium to be killed by a macrophage. In certain aspects, the cell infected with a bacterium is a macrophage. In other aspects, the bacterial cell is present within a macrophage phagosome.

Certain embodiments of the present invention are directed to methods for therapeutically treating a polyglutamine disorder in a human or non-human mammal in need thereof. The methods include administering to the human or non-human mammal a prokaryotic 20S proteasome or a portion thereof, and a pharmaceutically acceptable carrier. In certain aspects, the polyglutamine disorder is neurodegenerative disorder. In other aspects, the polyglutamine disorder is selected from the group consisting of: Huntington's disease, spinocerebellar ataxia type 1, spinocerebellar ataxia type 2, spinocerebellar ataxia type 3, spinocerebellar ataxia type 6, spinocerebellar ataxia type 7, dentatorubral-pallidoluysian atrophy, spinobulbar muscular atrophy, oculopharyngeal muscular dystrophy, and Huntington's disease-like Type 2.

Embodiments of the present invention are directed to pharmaceutical compositions for therapeutically treating a bacterial infection and pharmaceutical compositions for therapeutically treating one or more symptoms associated with a bacterial infection. Symptoms associated with a bacterial infection can include chest pain, non-productive coughing, coughing up blood, coughing up sputum, weakness, fatigue, weight loss, loss of appetite, chills, fever, night sweats and the like. The pharmaceutical compositions include a glutamine-glutamine dipeptide, a glutamine-glutamine-glutamine tripeptide or a polypeptide comprising a polyQ domain, and a pharmaceutically acceptable carrier. In certain aspects, the dipeptide, tripeptide or polypeptide includes an amino-terminal blocking moiety, such as N-acetyl, N-formyl, tert-butylcarbonyl, para-nitrophenylfornate and the like. In other aspects, the dipeptide, tripeptide or polypeptide includes a carboxy-terminal group that reacts with an active site of a proteasome, such as boronic acid, aldehyde, vinyl sulfone, epoxyketone, a beta lactone ring and the like.

Certain embodiments of the invention relate to methods of identifying and synthesizing additional inhibitors of microbial proteasomes that are more potent and/or equally selective as the triglutamine or diglutamine sequences described herein in inhibiting bacterial (e.g., tuberculosis) proteasomes. In one aspect, microbial proteasome inhibitors are peptide sequences that can optionally be attached to moieties such as boronic acid, aldehyde, vinyl sulfone or epoxyketone moieties, or to a beta lactone ring.

It will be recognized by the person of ordinary skill in the art that the compounds, compositions and methods disclosed herein provide significant advantages over prior technology. Compounds, compositions and methods can be designed or selected to relieve and/or alleviate symptoms in a patient suffering from one or more bacterial infections, one or more disorders associated with a bacterial infection and/or one or more diseases or disorders associated with polyQ-containing peptides and/or proteins. These and other aspects and examples are described below. Other features and advantages of the invention will be apparent from the following detailed description and claims.

### BRIEF DESCRIPTION OF THE DRAWINGS

The foregoing and other features and advantages of the present invention will be more fully understood from the following detailed description of illustrative embodiments taken in conjunction with the accompanying drawings.

FIGS. 1A-1D depict hydrolysis of bKKQ₁₀KK (SEQ ID NO:5) by the trypsin-like active site of the eukaryotic proteasome. A) 80 μM bKKQ₁₀KK (SEQ ID NO:5) was incubated with mammalian (rabbit) 20S proteasomes with or without PA28αβ, 0.02% SDS. B) 35 μM bKKQ₁₀KK (SEQ ID NO:5) was incubated with yeast wild type and ‘open-gate’ (α3ΔN) proteasomes. C) Mammalian 20S proteasomes were treated with inhibitors specific to each active site. D) After inactivation, the proteasomes were further incubated with bKKQ₁₀KK (SEQ ID NO:5).

FIG. 2 graphically depicts rapid cleavage of the flanking RRGRR (SEQ ID NO:6) in Q₂₀RRGRR (SEQ ID NO:7) by eukaryotic proteasomes. 35 μM of each of the following peptides bKKQ₁₀KK (SEQ ID NO:8), bKKQ₂₀KK (SEQ ID NO:9), N-extended-KK-Q₂₀-KK (SEQ ID NO:10), Q₂₀RRGRR (SEQ ID NO:7) and a control 20-mer peptide QATVGDINTERPGMLDFTGK (SEQ ID NO:12), lacking any repeat sequence were incubated with 7.2 μg of rabbit 26S particles in a total volume of 40 μl. New amino groups formed were assayed.

FIGS. 3A-3D depict matrix-assisted laser desorption/ionization-time of flight mass spectrometry (MALDI-TOF) analyses of polyQ sequence cleavage. Reaction mixtures containing bKKQ₂₀KK (SEQ ID NO:9) alone (A) or with rabbit 26S proteasomes (B). Using excess of yeast ‘open-gate proteasomes’, which cleaved bKKQ₂₀KK (SEQ ID NO:9) faster than the rabbit 26S proteasomes, large amounts of Q₁₉KK (SEQ ID NO:13), Q₁₈KK (SEQ ID NO:14) and Q₁₇KK (SEQ ID NO:15) were generated (C). The precipitate formed was resuspended in 100% acetonitrile containing 1% trifluoroacetic acid (TFA) (D).

FIGS. 4A-4B depict multiple cleavages within the polyQ sequence by archaebacterial proteasomes. A) bKKQ₂₀KK (SEQ ID NO:9) (25 μM) was incubated with 0.6 μg of yeast or 250 ng of archaebacterial ‘open-gate’ 20S particles in a total volume of 50 μl. B) Products generated by the archaebacterial proteasomes were analyzed by MALDI-TOF.

FIGS. 5A-5C depict degradation of myoglobin and Q₃₅ myoglobin by yeast and archaebacterial proteasomes. Upon degradation of Q₃₅ myoglobin, yeast proteasomes spare but the archaebacterial proteasomes degrade the poly-Q sequence. Recombinant myoglobin (A) and myoglobin fused to Q₃₅ repeat (B) were incubated with yeast ‘open-gate’ proteasomes. Aliquots of the reaction were boiled in 1× lithium dodecyl sulfate (LDS) sample buffer (Invitrogen, Carlsbad, Calif.) containing 100 mM DTT and separated by SDS-PAGE and probed with either polyclonal antibody against myoglobin (A, B) or with monoclonal antibody against glutamine repeat (C). Without the proteasomes, Q₃₅ myoglobin formed SDS-resistant aggregates. Myoglobin and Q₃₅ myoglobin were also incubated with archaebacterial ‘open-gate’ proteasomes and probed similarly. For simplicity, Q₃₅-myoglobin probed with polyQ antibody alone is shown in C.

### DETAILED DESCRIPTION

In accordance with certain examples, compounds (e.g., peptides) that inhibit prokaryotic (e.g., bacterial) proteasomes are provided. Such compounds are useful for treating bacterial infections and disorders associated with bacterial infections.

Compounds provided herein are effective to inhibit bacterial proteasomes at least to the extent necessary for the effective treatment of a bacterial infection and/or of one or more disorders associated with a bacterial infection. While in certain examples the bacterial proteasome may be substantially inhibited such that little or no bacterial proteasome-mediated protein degradation occurs, in other examples the inhibition is at least sufficient to relieve and/or alleviate symptoms associated with a bacterial infection and/or a disorder associated with a bacterial infection.

As used herein, the terms peptide, polypeptide and oligopeptide refer to a chain of amino acids linked by amide (i.e., peptide) bonds. Peptides, polypeptides and oligopeptides are typically less than about 50 amino acids in length. The terms peptide, polypeptide and oligopeptide can also be used to refer to a portion of a protein. As used herein, the term protein refers to a chain of amino acids that is typically greater than 50 amino acids in length. In certain aspects of the invention, a peptide is a dipeptide or a tripeptide. That is, the peptide comprises two or three amino acid residues, respectively (e.g., a QQ or a QQQ sequence, respectively). In other aspects, peptides of the present invention comprise an amino-terminal blocking moiety. As used herein, the term blocking moiety includes, but is not limited to, N-acetyl (Ac), N-formyl, Boc (tert-butylcarbonyl), para-nitrophenylfornate (NMP), detectable moieties and the like. In still other aspects, peptides of the present invention comprise a carboxy-terminal “warhead”. As used herein, the term warhead refers to a functional group that reacts with an active site of the proteasome. Warheads include, but are not limited to, boronic acid, aldehyde, vinyl sulfone or epoxyketone moieties, a beta lactone ring and the like.

In certain aspects, peptides of the invention comprise a polyQ domain. A polyQ domain, as used herein, is intended to include, but is not limited to, peptides and/or proteins having Q-rich regions. A peptide and/or protein having a polyQ domain can have 2, 3, 4, 5, 6, 7, 8, 9, 10, 15, 20, 25, 30, 35, 40, 45 or more Q residues.

As used herein, bacteria include, but are not limited to, archaebacteria, acid-fast bacteria, gram positive bacteria, gram negative bacteria and the like. As used herein, acid-fast bacteria include, but are not limited to, Myobacterium tuberculosis, Myobacterium avium, Myobacterium leprae, Mycobacterium ulcerans and the like.

As used herein, archaebacteria include, but are not limited to, methanogens (e.g., methanococcus jannaschii), halophiles (e.g., Actinopolyspora halophila, Ectothiorhodospira halochloris, Halobacterium salinarium and the like), thermophiles (e.g., Thermoplasma acidophilum, Thermus aquaticus, Pyrolobus fumarii, Sulfolobus acidocaldarius, Thermocrinis ruber and the like Pyrococcus furiosis) and the like.

As used herein, gram positive bacteria include, but are not limited to, Actinomedurae, Actinomyces israelii, Bacillus anthracis, Bacillus cereus, Clostridium botulinum, Clostridium difficile, Clostridium perfringens, Clostridium tetani, Corynebacterium, Enterococcus faecalis, Listeria monocytogenes, Nocardia, Propionibacterium acnes, Staphylococcus aureus, Staphylococcus epiderm, Streptococcus mutans, Streptococcus pneumoniae and the like.

As used herein, gram negative bacteria include, but are not limited to, Afipia felis, Bacteriodes, Bartonella bacilliformis, Bortadella pertussis, Borrelia burgdorferi, Borrelia recurrentis, Brucella, Calymmatobacterium granulomatis, Campylobacter, Escherichia coli, Francisella tularensis, Gardnerella vaginalis, Haemophilus aegyptius, Haemophilus ducreyi, Haemophilus influenziae, Heliobacter pylori, Legionella pneumophila, Leptospira interrogans, Neisseria meningitidia, Porphyromonas gingivalis, Providencia sturti, Pseudomonas aeruginosa, Salmonella enteridis, Salmonella typhi, Serratia marcescens, Shigella boydii, Streptobacillus moniliformis, Streptococcus pyogenes, Treponema pallidum, Vibrio cholerae, Yersinia enterocolitica, Yersinia pestis and the like.

As used herein, other bacteria not falling into the other three categories include, but are not limited to, Bartonella henseiae, Chlamydia psittaci, Chlamydia trachomatis, Coxiella burnetii, Mycoplasma pneumoniae, Rickettsia akari, Rickettsia prowazekii, Rickettsia rickettsii, Rickettsia tsutsugamushi, Rickettsia typhi, Ureaplasma urealyticum, Diplococcus pneumoniae, Ehrlichia chafensis, Enterococcus faecium, Meningococci and the like.

In accordance with certain example, peptides are provided to selectively inhibit bacterial proteasomes. Peptides of the present invention may be assembled sequentially from individual amino acids or by linking suitable small peptide fragments. In sequential assembly, the peptide chain is extended stepwise, starting at the C-terminus, by one amino acid per step. In fragment coupling, fragments of different lengths can be linked together, and the fragments can also be obtained by sequential assembly from amino acids or by fragment coupling of still shorter peptides.

In both sequential assembly and fragment coupling it is necessary to link the units (e.g., amino acids, peptides, compounds and the like) by forming an amide linkage, which can be accomplished via a variety of enzymatic and chemical methods. The methods described herein for formation of peptidic amide linkages are also suitable for the formation of non-peptidic amide linkages.

Chemical methods for forming the amide linkage are described in detail in standard references on peptide chemistry, including Muller, Methoden der organischen Chemie Vol. XV/2, 1-364, Thieme Verlag, Stuttgart, (1974); Stewart and Young, Solid Phase Peptide Synthesis, 31-34 and 71-82, Pierce Chemical Company, Rockford, Ill. (1984); Bodanszky et al., Peptide Synthesis, 85-128, John Wiley & Sons, New York, (1976); Practice of Peptide Synthesis, M. Bodansky, A. Bodansky, Springer-Verlag, 1994 and other standard works in peptide chemistry, incorporated herein by reference in their entirety for all purposes. Methods include the azide method, the symmetric and mixed anhydride method, the use of in situ generated or preformed active esters, the use of urethane protected N-carboxy anhydrides of amino acids and the formation of the amide linkage using coupling reagents, such as dicyclohexylcarbodiimide (DCC), diisopropylcarbodiimide (DIC), 1-ethoxycarbonyl-2-ethoxy-1,2-dihydroquinoline (EEDQ), pivaloyl chloride, 1-ethyl-3-(3-dimethylaminopropyl)carbodiimide hydrochloride (EDCI), n-propane-phosphonic anhydride (PPA), N,N-bis(2-oxo-3-oxazolidinyl)amido phosphoryl chloride (BOP—Cl), bromo-tris-pyrrolidinophosphonium hexafluorophosphate (PyBrop), diphenylphosphoryl azide (DPPA), Castro's reagent (BOP, PyBop), O-benzotriazolyl-N,N,N′,N′-tetramethyluronium salts (HBTU), O-azabenzotriazolyl-N,N,N′,N′-tetramethyluronuim salts (TATU), diethylphosphoryl cyanide (DEPCN), 2,5-diphenyl-2,3-dihydro-3-oxo-4-hydroxythiophene dioxide (Steglich's reagent; HOTDO), 1,1′-carbonyldiimidazole (CDI) and the like. The coupling reagents can be employed alone or in combination with additives such as N,N-dimethyl-4-aminopyridine (DMAP), N-hydroxy-benzotriazole (HOBt), N-hydroxybenzotriazine (HOOBt), N-hydroxysuccinimide (HOSu), 2-hydroxypyridine and the like.

In accordance with certain examples, methods and compounds for the therapeutic treatment of diseases and disorders associated with polyQ-containing peptides and/or proteins are disclosed. While in certain examples the polyQ-containing peptides and/or proteins may be substantially degraded such that little or none of polyQ-containing peptides and/or proteins remain, in other examples the inhibition is at least sufficient to relieve and/or alleviate one or more symptoms associated with a polyQ-associated disease and/or disorder. In certain embodiments, alpha and/or beta subunits or portions thereof are provided to degrade polyQ-containing polypeptides and/or proteins to alleviate and/or reduce one or more symptoms associated with a polyQ-associated disease and/or disorder.

PolyQ-associated diseases and disorders are typically characterized as neurodegenerative diseases and disorders and may involve protein and/or peptide aggregation and/or the formation of inclusions. As used herein, polyQ-associated diseases and disorders include, but are not limited to, Huntington's disease, spinocerebellar ataxia types 1, 2, 3, 6 and 7, dentatorubral-pallidoluysian atrophy, spinobulbar muscular atrophy, oculopharyngeal muscular dystrophy, Huntington's disease-like Type 2 and the like.

Pharmaceutical Compositions

In accordance with certain examples, the compounds of the present invention can be incorporated into pharmaceutical compositions suitable for administration. Such compositions typically comprise the compound and a pharmaceutically acceptable carrier. As used herein the term “pharmaceutically acceptable carrier” is intended to include, but is not limited to, any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811, incorporated herein by reference in its entirety for all purposes.

Nasal compositions generally include nasal sprays and inhalants. Nasal sprays and inhalants can contain one or more active components and excipients such as preservatives, viscosity modifiers, emulsifiers, buffering agents and the like. Nasal sprays may be applied to the nasal cavity for local and/or systemic use. Nasal sprays may be dispensed by a non-pressurized dispenser suitable for delivery of a metered dose of the active component. Nasal inhalants are intended for delivery to the lungs by oral inhalation for local and/or systemic use. Nasal inhalants may be dispensed by a closed container system for delivery of a metered dose of one or more active components.

In one embodiment, nasal inhalants are used with an aerosol. This is accomplished by preparing an aqueous aerosol, liposomal preparation or solid particles containing the compound. A non-aqueous (e.g., fluorocarbon propellant) suspension could be used. Sonic nebulizers may be used to minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Therapeutic Methods

One aspect of the invention pertains to methods of modulating bacterial proteasomes for therapeutic purposes. Accordingly, in exemplary embodiments, the modulatory method of the invention involves contacting a cell infected by a bacterium, contacting a bacterium and/or contacting an infected subject (i.e., a bacterial host, e.g., a mammal) with an agent that inhibits a bacterial proteasome. In one aspect, the compound inhibits a bacterial proteasome while only minimally inhibiting a eukaryotic (e.g., mammalian) proteasome. Methods of modulating bacterial proteasome activity can be performed in vitro (e.g., by culturing a cell with the agent) or, alternatively, in vivo (e.g., by administering the agent to a subject). As such, the present invention provides methods of treating an individual afflicted with a bacterial infection and/or a disease or disorder associated with a bacterial infection. Examples of such disorders are described herein. In one embodiment, the method involves administering to a subject (i.e., a bacterial host) a compound (e.g., a compound identified by a screening assay described herein), or combination of compounds that inhibits a prokaryotic proteasome.

One embodiment of the present invention involves a method for treatment of a bacterial infection or disorder associated with a bacterial infection which includes the step of administering a therapeutically effective amount of an agent which inhibits a bacterial proteasome to a subject. As defined herein, a therapeutically effective amount of agent (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an inhibitor can include a single treatment or, preferably, can include a series of treatments. It will also be appreciated that the effective dosage of inhibitor used for treatment may increase or decrease over the course of a particular treatment. Changes in dosage may result from the results of diagnostic assays as described herein.

Screening Assays

In one embodiment, the present invention is directed to methods of screening inhibitors of prokaryotic (e.g., bacterial) proteasomes. In one embodiment, bacterial proteasomes can be isolated by standard biochemical approaches in order to screen for test compounds. In one aspect, Mycobacterium smegmatis can be used as it may be grown easily and has a proteasome that is virtually identical to the proteasome of Mycobacterium tuberculosis (see Knipfer and Shrader (1997) Mol. Microbiol. 25:375, incorporated herein by reference in its entirety for all purposes). In another aspect, the alpha and beta subunits of the 20S Mycobacterium tuberculosum gene may be cloned, and large amounts of the proteasomes of Thermoplasma acidophilum may be generated in E. coli (Kim et al. (1995) J. Biol. Chem. 270:29799, incorporated herein by reference in its entirety for all purposes). Isolations of proteasomes from rabbit, bovine, or human tissues are now standard methods, and the proteasomes from different tissues and from different mammalian species are extremely similar. The standard assays described above would allow the investigator to identify peptide sequences that are most potent against the microbial proteasomes, but do not affect the mammalian particles. Then, use of classic approaches of medicinal chemistry could be used to modify these peptide sequences to enhance potency, cell uptake, or resistance to destruction by other enzymes, as was done in the development of other proteasome inhibitors, such as VELCADE™ (PS341) (Millennium Pharmaceuticals, Cambridge, Mass.). Efficacy can be evaluated and optimized (e.g., to increase uptake of the drug by cells) using standard microbicidal assays, using methods known in the art (e.g., those taught by Darwin et al. (2003) Science 302:1963, incorporated herein by reference in its entirety for all purposes).

The ability of one or more peptides to inhibit a mammalian proteasome can be assayed using standard fluorogenic substrates for each active site (see Kisselev and Goldberg (2001) supra; Kisselev et al. (2002) J. Biol. Chem. 25:22260; Kisselev et al. (2003) J. Biol. Chem. 278:35869, incorporated herein by reference in their entirety for all purposes). The ability of one or more peptides to inhibit a eukaryotic proteasome can be determined by examining the three active sites in the eukaryotic proteasome (Venkatraman et al., supra, incorporated herein by reference in its entirety for all purposes, and data set forth below). The substrate succinyl Leu-Leu-Val-Tyr-mca (SEQ ID NO:16) can be used, for example, to establish efficacy for the microbial proteasomes (see Knipfer and Shrader (1997) Mol. Microbiol. 25:375; Akopian et al. (1997) J. Biol. Chem. 272:1791, incorporated herein by reference in their entirety for all purposes). Standard methods using fluorogenic peptides are also available to confirm a lack of potency against the mammalian proteasome, as would be desirable for an antimicrobial drug (see Kisselev et al. (2003) supra or Venkatraman et al., supra, incorporated herein by reference in their entirety for all purposes).

In another embodiment, an assay is a cell-based assay comprising contacting a cell expressing a proteasome with a test compound and determining the ability of the test compound to modulate (e.g. inhibit) the activity of the proteasome. Determining the ability of the test compound to modulate the activity of a proteasome can be accomplished, for example, by determining the ability of a compound to inhibit proteasomal activity in cells or cell extracts to cause accumulation of a short-lived model protein that is normally degraded rapidly by the proteasome (e.g., IκB or ubiquitin-pro-β-galactosidase) (See Kisselev and Goldberg (2001) supra, incorporated herein by reference in its entirety for all purposes).

In yet another embodiment, an assay of the present invention is a cell-free assay in which a proteasome or a biologically active portion thereof is contacted with a test compound and the ability of the test compound to bind to the proteasome or a biologically active portion of the proteasome is determined. Binding of the test compound to a proteasome can be determined either directly or indirectly as described above.

In one aspect, peptide sequences useful for inhibiting a bacterial proteasome can be identified using peptide or inhibitor libraries by methods known to those of skill in the art (Harris et al. (2001) Chemistry and Biology 8:1131; Bogyo et al. (1997) Proc. Natl. Acad. Sci. USA 94:6629; Kisselev et al. (2003) supra, incorporated herein by reference in their entirety for all purposes). These methods can be used to predict measure binding affinity of certain residues in certain positions of the peptide sequences. While not intending to be bound by theory, tripeptides containing one or more of the following residues before the warhead should bind poorly to the mammalian proteasome. Peptides of the invention include, but are not limited to, glutamine, histidine, glycine, proline, serine or threonine residues in the P1 position; proline, tyrosine, phenylalanine, aspartate, or isoleucine residues in the P2 position; tryptophan, glycine, proline, seine, threonine or tryptophan residues in the P3 position; a serine residue in the P4 position.

Test compounds (e.g., peptides) can be isolated from cells or tissue sources using standard protein purification techniques, be produced by recombinant DNA techniques or synthesized chemically by standard methods. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection (Lam, K. S. (1997) Anticancer Drug Dis. 12:145, incorporated herein by reference in its entirety for all purposes).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233 (incorporated herein by reference in their entirety for all purposes).

Libraries of compounds may be presented in solution (e.g., Houghten (1992) Biotechniques 13:412), or on beads (Lam (1991) Nature 354:82), chips (Fodor (1993) Nature 364:555), bacteria (Ladner, U.S. Pat. No. 5,223,409), spores (Ladner, U.S. Pat. No. 5,223,409), plasmids (Cull et al. (1992) Proc. Natl. Acad. Sci. USA 89:1865) or on phage (Scott and Smith (1990) Science 249:386; Devlin (1990) Science 249:404; Cwirla et al. (1990) Proc. Natl. Acad. Sci. USA 87:6378; Felici (1991) J. Mol. Biol. 222:301; and Ladner, supra) (incorporated herein by reference in their entirety for all purposes).

Recombinant Expression Vectors and Host Cells

Another aspect of the invention pertains to vectors, such as expression vectors, containing a nucleic acid encoding one or more polyQ peptides or one or more alpha and/or beta subunits of a prokaryotic 20S proteasome (e.g., an M. tuberculosum proteasome). As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid encoding a peptide and/or protein of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990) (incorporated herein by reference in its entirety for all purposes). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, (e.g., polyQ peptides, alpha and/or beta subunits of a prokaryotic 20S proteasome, and the like).

The recombinant expression vectors of the invention can be designed for expression of polyQ peptides and/or alpha and/or beta subunits of a prokaryotic 20S proteasome in prokaryotic or eukaryotic cells. For example, polyQ peptides and/or alpha and/or beta subunits of a prokaryotic 20S proteasome can be expressed in bacterial cells such as E. coli, insect cells (using baculovirus expression vectors), yeast cells, amphibian cells or mammalian cells. Suitable host cells are discussed further in Goeddel, Gene Expression Technology: Methods in Enzymology 185, Academic Press, San Diego, Calif. (1990) (incorporated herein by reference in its entirety for all purposes). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion proteins. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40, incorporated herein by reference in its entirety for all purposes), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315, incorporated herein by reference in its entirety for all purposes) and pET 11d (Studier et al., Gene Expression Technology: Methods in Enzymology 185, Academic Press, San Diego, Calif. (1990) 60-89, incorporated herein by reference in its entirety for all purposes). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128, incorporated herein by reference in its entirety for all purposes). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111, incorporated herein by reference in its entirety for all purposes). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., (1987) Embo J. 6:229), pMFa (Kurjan and Herskowitz, (1982) Cell 30:933), pJRY88 (Schultz et al., (1987) Gene 54:113), pYES2 (Invitrogen Corporation, San Diego, Calif.), and picZ (InVitrogen Corp, San Diego, Calif.) (each incorporated herein by reference in its entirety for all purposes).

Alternatively, polyQ peptides and/or alpha and/or beta subunits of a prokaryotic 20S proteasome can be expressed in insect cells using baculovirus expression vectors. Baculovirus vectors available for expression of proteins in cultured insect cells (e.g., Sf 9 cells) include the pAc series (Smith et al. (1983) Mol. Cell. Biol. 3:2156, incorporated herein by reference in its entirety for all purposes) and the pVL series (Lucklow and Summers (1989) Virology 170:31, incorporated herein by reference in its entirety for all purposes).

In yet another embodiment, a nucleic acid of the invention is expressed in mammalian cells using a mammalian expression vector. Examples of mammalian expression vectors include pCDM8 (Seed, B. (1987) Nature 329:840, incorporated herein by reference in its entirety for all purposes) and pMT2PC (Kaufman et al. (1987) EMBO J. 6:187, incorporated herein by reference in its entirety for all purposes). When used in mammalian cells, the expression vector's control functions are often provided by viral regulatory elements. For example, commonly used promoters are derived from polyoma, adenovirus 2, cytomegalovirus and Simian virus 40. For other suitable expression systems for both prokaryotic and eukaryotic cells see Current Protocols in Molecular Biology. John Wiley & Sons, Inc., 1998, incorporated herein by reference in its entirety for all purposes.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268, incorporated herein by reference in its entirety for all purposes), lymphoid-specific promoters (Calame and Eaton (1988) Adv. Immunol. 43:235, incorporated herein by reference in its entirety for all purposes), in particular promoters of T cell receptors (Winoto and Baltimore (1989) EMBO J. 8:729, incorporated herein by reference in its entirety for all purposes) and immunoglobulins (Banerji et al. (1983) Cell 33:729; Queen and Baltimore (1983) Cell 33:741, incorporated herein by reference in their entirety for all purposes), neuron-specific promoters (e.g., the neurofilament promoter; Byrne and Ruddle (1989) Proc. Natl. Acad. Sci. USA 86:5473, incorporated herein by reference in its entirety for all purposes), pancreas-specific promoters (Edlund et al. (1985) Science 230:912, incorporated herein by reference in its entirety for all purposes), and mammary gland-specific promoters (e.g., milk whey promoter; U.S. Pat. No. 4,873,316 and European Application Publication No. 264,166, incorporated herein by reference in its entirety for all purposes). Developmentally regulated promoters are also encompassed, for example the murine hox promoters (Kessel and Gruss (1990) Science 249:374, incorporated herein by reference in its entirety for all purposes) and the α-fetoprotein promoter (Campes and Tilghman (1989) Genes Dev. 3:537, incorporated herein by reference in its entirety for all purposes).

Another aspect of the invention pertains to host cells into which a recombinant expression vector of the invention has been introduced, containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, host cells can be bacterial cells such as E. coli, insect cells, yeast, Xenopus cells, or mammalian cells (such as Chinese hamster ovary cells (CHO), African green monkey kidney cells (COS), or fetal human cells (293T)). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation or microinjection. Suitable methods for transforming or transfecting host cells are described in the art (e.g., Current Protocols in Molecular Biology. John Wiley & Sons, Inc., 1998, incorporated herein by reference in its entirety for all purposes), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Suitable selectable markers include those which confer resistance to drugs, such as G418, hygromycin and methotrexate. Nucleic acid encoding a selectable marker can be introduced into a host cell on the same vector as that encoding a detectable translation product or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) polyQ peptides and/or alpha and/or beta subunits of a prokaryotic 20S proteasome. Accordingly, the invention further provides methods for producing the polyQ peptides and/or alpha and/or beta subunits of a prokaryotic 20S proteasome using the host cells of the invention. In one embodiment, the method comprises culturing the host cell of invention (into which a recombinant expression vector encoding a detectable translation product has been introduced) in a suitable medium such that a detectable translation product is produced. In another embodiment, the method further comprises isolating the polyQ peptides and/or alpha and/or beta subunits of the 20S proteasome from the medium or the host cell.

This invention is further illustrated by the following examples, which should not be construed as limiting. The contents of all references, patents and published patent applications cited throughout this application are hereby incorporated by reference in their entirety for all purposes.

### EXAMPLE I

### Mammalian Proteasomes Cleave bKKQ₁₀KK (SEQ ID NO:8) to bKKQ and Q₉KK (SEQ ID NO:17)

The ability of purified mammalian 20S and 26S proteasomes to digest peptides containing 10-30 Q residues with flanking basic amino acids that ensured polyQ solubility was explored. Because these model substrates could be maintained soluble at high concentrations, it was possible to obtain sufficient amounts of products to analyze the sites of cleavage. To ensure that these substrates could enter the 20S proteasomes, various conditions were used that favored gate opening in the α-ring, including mammalian 26S proteasomes, 20S particles treated with PA28αβ or SDS, or mutant yeast proteasomes (α3ΔN) where this gate is constitutively open (Groll et al., supra, incorporated herein by reference in its entirety for all purposes). These conditions allowed the normal requirements for ubiquitination, unfolding and translocation of the substrate by the 19S complex to be bypassed in order to specifically test whether the proteolytic sites can cleave a polyQ chain once it has been translocated into the 20S particle.

Whether these particles could digest a polyQ chain was tested. Eukaryotic 26S and 20S proteasomes failed to cut within stretches of 9-29 residues in peptides. While digesting a myoglobin-Q₃₅ fusion protein, the proteasomes spared the polyQ sequence. In contrast, archaebacterial proteasomes, whose 14 active sites are less specific, rapidly digested such polyQ repeats. Therefore, when degrading polyQ proteins, eukaryotic proteasomes must release polyQ-containing fragments for further hydrolysis by unidentified peptidases.

To determine if the results obtained were due to the specificity of the active sites in eukaryotic proteasomes, similar studies were carried with 20S proteasomes from the archaebacteria, Thermoplasma acidophilum, which have a single type of active site of broad specificity. These proteasomes are the evolutionary precursors of the eukaryotic particles and are similar in structure, subunit number, and catalytic mechanism; they cleave all the fluorogenic substrates of the eukaryotic proteasomes and generate products of similar size (Akopian et al., supra; Kisselev et al. (1998) J. Biol. Chem. 273:1982, incorporated herein by reference in their entirety for all purposes). The data presented herein demonstrates that eukaryotic proteasomes, unlike those of the archaea, cannot digest a polyQ chain.

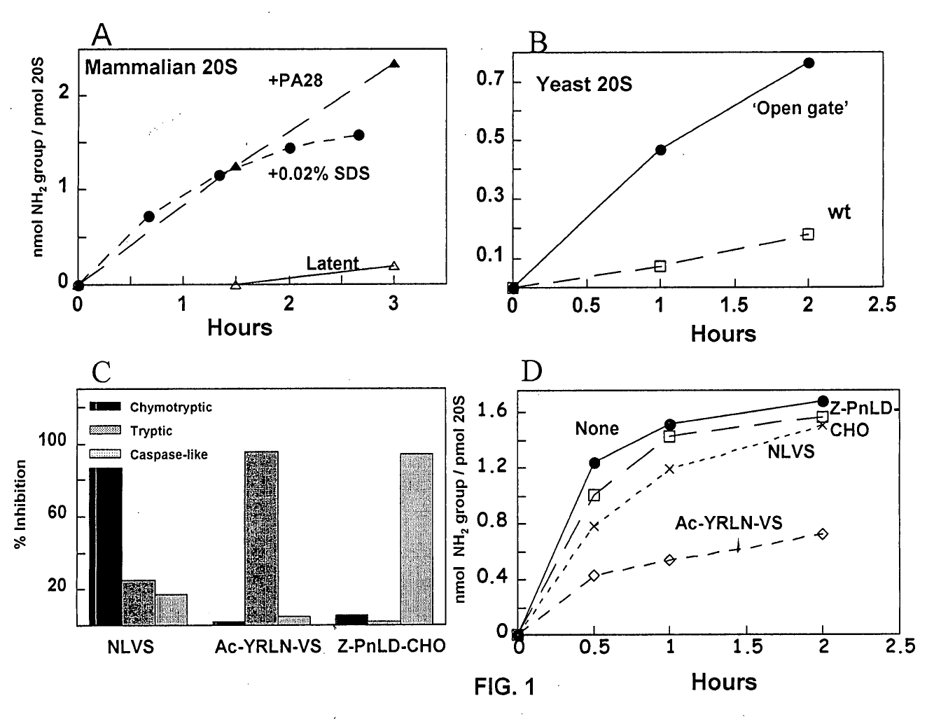

To test whether 20S proteasomes could cleave within a polyQ sequence, a peptide containing 10 Q-residues flanked at each end by two lysine residues to enhance solubility (bKKQ₁₀KK) (SEQ ID NO:8) and biotin on the α amino group was initially used as a substrate. Rabbit 20S proteasomes, when purified in their latent state, were inactive against bKKQ₁₀KK (SEQ ID NO:8) (FIG. 1A) or against standard fluorogenic peptide substrates, unless PA28αβ, or 0.02% SDS was added to cause opening of the channel in the α-ring. Similarly, a mutant form (α3ΔN) of the yeast proteasome with an N-terminal deletion in the α3 subunit, which causes ‘gate-opening’, hydrolyzed bKKQ₁₀KK (SEQ ID NO:8), approximately ten-fold faster than the wild-type (FIG. 1B). Thus, entry of this peptide and its cleavage depends on the presence of an ‘open-gate’ in the α-ring.

To determine where bKKQ₁₀KK (SEQ ID NO:8) was cleaved by the proteasomes, products from a four hour reaction were analyzed by MALDI-TOF MS. With either PA28αβ or 0.02% SDS present, only two major fragments, corresponding in mass to bKKQ and Q₉KK (SEQ ID NO: 17), were generated (Table 1). Small amounts of another peptide corresponding in mass to Q₈KK (SEQ ID NO:18) were also generated, which, without intending to be bound by theory, were likely produced by a similar cleavage after bKKQ in the low amount of bKKQ₉KK (SEQ ID NO:19) present as a contaminant in bKKQ₁₀KK (SEQ ID NO:8). Thus, the proteasomes made a single cut after the initial Q-residue adjacent to the N-terminal flanking sequence, and no further cleavage was observed within the Q₉KK (SEQ ID NO:17), even after long incubation when the bKKQ₁₀KK (SEQ ID NO:8) was consumed.

TABLE 1

| | Calculated Mass | Observed Mass |
|---|---|---|
| SUBSTRATE | | |
| bKKQ₁₀KK | 2056 | 2038 |
| (SEQ ID NO: 8) | | |
| PRODUCTS | | |
| bKKQ | 644 | 644 |
| Q₉KK | 1428 | 1428 |
| (SEQ ID NO: 17) | | |
| Q₈KK (minor) | 1299 | 1299 |
| (SEQ ID NO: 18) | | |

CLEAVAGES: bKKQ^{↓}Q₉KK

### EXAMPLE II

### The ‘Trypsin-Like’ Active Site Cleaves after the N-Terminal bKKQ

To identify which active site of the proteasome cleaved after bKKQ, inhibitors that selectively block the individual active sites were used. SDS-activated 20S mammalian proteasomes were treated with the irreversible inhibitors, 4-hydroxy-5-iodo-3-nitrophenylacetyl-Leu-Leu-vinyl sulfone (NLVS); (Bogyo et al., supra, incorporated herein by reference in its entirety for all purposes), which is specific for the chymotrypsin-like site; Acetyl-Tyr-Arg-Leu-Asn-vinyl sulfone (SEQ ID NO:20) (Ac-YRLN-VS), which reacts primarily with the trypsin-like site (Nazif et al. (2001) Proc. Natl. Acad. Sci. USA 98:2967, incorporated herein by reference in its entirety for all purposes); or with the reversible inhibitor of the caspase-like site, Z-Pro-norLeu-Asp-aldehyde (Z-PnLD-CHO) (Kisselev et al. (2003) supra, incorporated herein by reference in its entirety for all purposes). The extent of inhibition of the different sites was assayed using fluorogenic substrates specific for each active site. Each inhibitor caused an 85%-95% decrease in the individual peptidase activities without significantly affecting the other two (FIG. 1C). After inactivation of each type of active site, the proteasomes were incubated with bKKQ₁₀KK. Treatment with Ac-YRLN-VS (SEQ ID NO:20) reduced bKKQ₁₀KK (SEQ ID NO:8) cleavage by about 70% (FIG. 1D), while other inhibitors had no significant effect. Thus, cleavage after bKKQ was due to the activity of the trypsin-like site, presumably because of the two-lysine residues preceding the initial Q residue.

### EXAMPLE III

### Slow Cleavage of Q₂₀-Containing Peptides by 26S Proteasomes

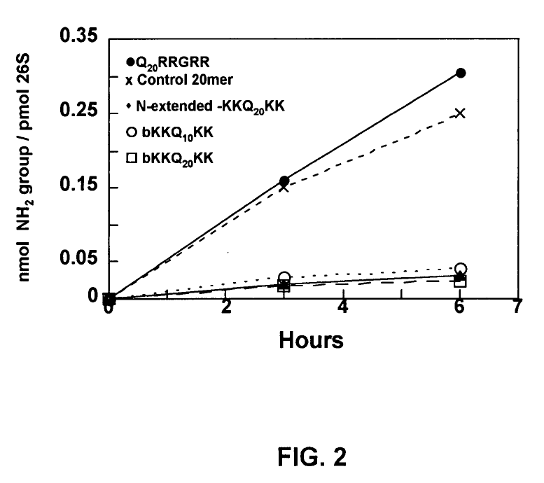

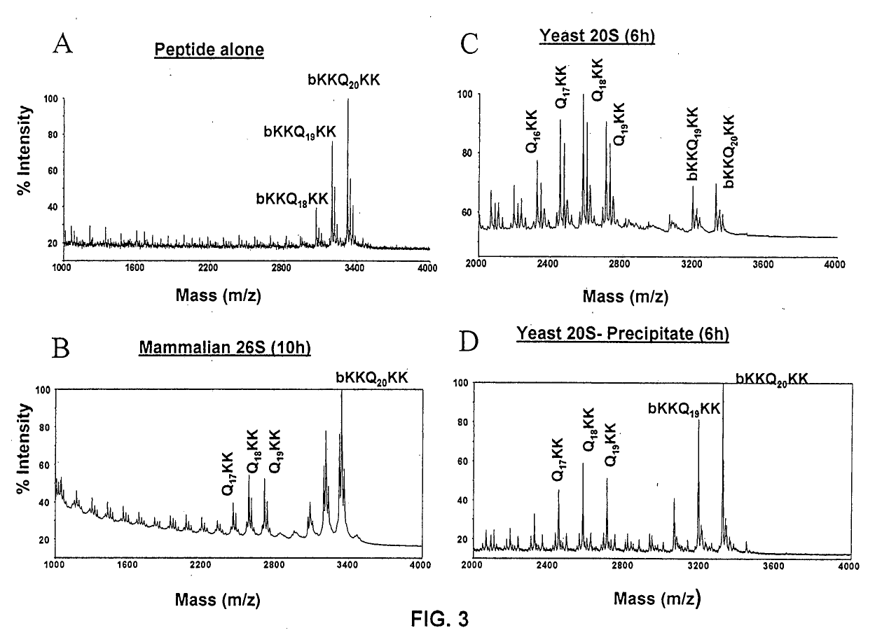

The capacity of purified 26S proteasomes to hydrolyze various polyQ peptides was next examined. Unlike 20S particles, native 26S particles, if provided ATP, can degrade many peptides and some proteins without ubiquitination (Cascio et al. (2001) Embo J. 20:2357; Kisselev et al., 1999, supra, incorporated herein by reference in their entirety for all purposes). Product analysis showed that the 26S proteasomes, like the ‘activated’ 20S particles, cleaved bKKQ₁₀KK (SEQ ID NO:8) to yield primarily bKKQ and Q₉KK (SEQ ID NO:17). No additional cleavages occurred within the Q₉KK (SEQ ID NO:17) even after a 24 hour incubation. To test whether longer polyQ sequences influenced the rate or sites of cleavage, similar experiments utilized a Q₂₀ (SEQ ID NO: 30) sequence flanked by two lysine residues on each end and biotin on the α amino group, bKKQ₂₀KK (SEQ ID NO:9), or the same peptide with a 15-residue N-extension, GAPVPYPDPLEPRGG (SEQ ID NO:21), in place of the biotin. These 20 Q-peptides were cleaved more slowly than control peptides of similar size that lacked repeat sequences (e.g., QATVGDINTERPGMLDFTGK (SEQ ID NO:22); FIG. 2), but were cleaved at similar rates to homologous peptides containing 10 or 30Qs, bKKQ₁₀KK (SEQ ID NO:8) and bKKQ₃₀KK (SEQ ID NO:23). Because proteasomes cut bKKQ₁₀KK (SEQ ID NO:8) only after bKKQ, these data indicated that the only site of cleavage in bKKQ₂₀KK (SEQ ID NO:9) was also after the bKKQ. One complication in experiments with Q₂₀KK (SEQ ID NO:24) peptides was that these substrates were not homogenous, since it proved impossible by standard methods to prepare them free of contaminating homolog peptides lacking one or two glutamine residues (FIG. 3A and Table 2). However, these shorter contaminants were degraded by proteasomes to yield similar products. Accordingly, when products from bKKQ₂₀KK (SEQ ID NO:9) were analyzed, peaks with mass units primarily corresponding to Q₁₉KK (SEQ ID NO:13) and Q₁₈KK (SEQ ID NO:14) (as well as trace amounts of Q₁₇KK (SEQ ID NO:15); FIGS. 3B-3D and Table 2) were detected. These results confirmed cleavages after bKKQ. Without intending to be bound by theory, Q₁₈KK (SEQ ID NO:14) was likely generated by a similar cleavage of the bKKQ₁₉KK (SEQ ID NO:1), and the trace amounts of Q₁₇KK (SEQ ID NO:15) was likely generated from a similar cleavage in the contaminating bKKQ₁₈KK (SEQ ID NO:2).

When the N-terminal biotin in bKKQ₂₀KK (SEQ ID NO:9) was replaced by a 15 residue sequence, GAPVPYPDPLEPRGG (SEQ ID NO:21), this longer substrate was cleaved by 26S proteasomes to give primarily GAPVPYPDPLEPRGGKKQ (SEQ ID NO:3) and Q₁₉KK (SEQ ID NO:13). Thus, despite the 15-residue N-extension, the primary cleavage still occurred after the KKQ, without significant cleavages within the long Q₁₉ repeat, even during a 24 hour incubation. The same products were also generated in similar experiments using SDS-activated 20S mammalian proteasomes. Increasing polyQ length did not add sites for proteasomal hydrolysis.

TABLE 2

| | Calculated Mass | Observed Mass |
|---|---|---|
| SUBSTRATE bKKQ₂₀KK (SEQ | | |
| ID NO: 9) | | |
| bKKQ₂₀KK (SEQ ID NO: 9) (major) | 3337 | 3319¹ |
| bKKQ₁₉KK (SEQ ID NO: 1) | 3209 | 3191¹ |
| (contaminant) | | |
| bKKQ₁₈KK (SEQ ID NO: 2) (minor | 3081 | 3083² |
| contaminant) | | |

PRODUCTS OF MAMMALIAN 26S AND
YEAST 20S PROTEASOMES

| | | |
|---|---|---|
| Q₁₉KK (SEQ ID NO: 13) (major) | 2708 | 2730³ |
| Q₁₈KK (SEQ ID NO: 14) (major) | 2580 | 2580 |
| Q₁₇KK (SEQ ID NO: 15) (minor) | 2452 | 2452 |

CLEAVAGES: bKKQ^{↓}Q₁₉KK (SEQ ID NO: 9) and
bKKQ^{↓}Q₁₈KK (SEQ ID NO: 1)
(major); bKKQ^{↓}Q₁₇KK (SEQ ID NO: 2) (minor)
SUBSTRATE Q₂₀RRGRR (SEQ
ID NO: 7)

| | | |
|---|---|---|
| Q₂₀RRGRR (SEQ ID NO: 7) (major) | 3262 | 3310⁴ |
| Q₁₉RRGRR (SEQ ID NO: 4) | 3134 | 3160⁵ |
| (contaminant) | | |

PRODUCTS OF MAMMALIAN 26S PROTEASOME

| | | |
|---|---|---|
| Q₂₀RRG (SEQ ID NO: 25) (major) | 2950 | 2997⁶ |
| Q₁₉RRG (SEQ ID NO: 11) (major) | 2821 | 2869⁴ |

CLEAVAGES: Q₂₀RRG^{↓}RR (SEQ ID NO: 7) and Q₁₉RRG^{↓}RR
(SEQ ID NO: 4)
PRODUCTS OF YEAST 20S PROTEASOME

| | | |
|---|---|---|
| Q₂₀RR (SEQ ID NO: 26) (major) | 2893 | 2894⁷ |
| Q₁₉RR (SEQ ID NO: 27) (major) | 2769 | 2770⁸ |
| Q₁₉R (SEQ ID NO: 28) (minor) | 2608 | 2609⁷ |

CLEAVAGES: Q₂₀RR^{↓}GRR (SEQ ID NO: 7); Q₁₉RR^{↓}GRR
(SEQ ID NO: 4) (major);
Q₁₉R^{↓}RGRR (SEQ ID NO: 4) (minor)
¹Minus 18 mass units is likely due to loss of water.
²Additional 42 mass units corresponds to a disodium adduct (2x22) and deamidation of 2 glutamine (Q) residues (2x1).
³Additional 22 mass units correspond to a sodium adduct.
⁴Additional 48 mass units to a disodium adduct (2x22) and deamidation of 4 Q residues (4x1).
⁵Additional 26 mass units due to a sodium adduct and deamidation of 4 Qs.
⁶Additional 47 mass units due to disodium adduct (2x22) and deamidation of 3 Q (3x1).
^{3&7}Additional 2 and ⁸1 mass units are likely due to deamidation of two and one Q respectively.

### EXAMPLE IV

### 26S Proteasomes Rapidly Cleave Q₂₀RRGRR (SEQ ID NO:7) by Cutting Only within the RRGRR (SEQ ID NO:6)

Although 26S proteasomes cleaved these polyQ peptides slowly and only after the initial Q residues, they cleaved a Q₂₀ (SEQ ID NO: 30) sequence with RRGRR (SEQ ID NO:6) at its C-terminus 5 to 6-fold faster than other polyQ peptides (FIG. 2) studied and at similar rates to the 20-mer control peptide (though not as fast as YGGFLRRIRPKLK (SEQ ID NO:29), a 13-mer which is cleaved at least 2 times faster). Without intending to be bound by theory, these findings indicate that polyQ peptides with 20-30 residues can enter the 26S proteasomes at comparable rates as control peptides of similar size, and that their cleavage rates are determined by the nature of the flanking residues and independently of the length of the polyQ sequence.

The rapid cleavage of Q₂₀RRGRR (SEQ ID NO:7) by 26S proteasomes could occur by cuts within the RRGRR (SEQ ID NO:6) or the Q₂₀ (SEQ ID NO: 30) sequence. To decide between these possibilities, the reaction products were analyzed by MALDI-TOF. Product analyses showed that the rapid hydrolysis of Q₂₀RRGRR (SEQ ID NO:7) by mammalian 26S proteasomes was actually due to cleavages within the C-terminal flanking sequence, RRGRR (SEQ ID NO:6), since the primary product was Q₂₀RRG (SEQ ID NO:25) (Table 2). Some Q₁₉RRG (SEQ ID NO:11) was also generated, which was likely produced by similar cleavages in the contaminating Q₁₉RRGRR (SEQ ID NO:4) (Table 2). Thus, the 26S proteasomes spared the entire polyQ repeat in this substrate and cut only within the flanking RRGRR (SEQ ID NO:6).

### EXAMPLE V

### Like Mammalian Proteasomes, Yeast Proteasomes Fail to Degrade polyQ Sequences

Yeast and mammalian proteasomes are very similar in structure and peptidase activities. The yeast 20S particles also cleaved bKKQ₂₀KK (SEQ ID NO:9) to bKKQ and Q₁₉KK (SEQ ID NO:13), as well as Q₁₈KK (SEQ ID NO:14) from the contaminating bKKQ₁₉KK (SEQ ID NO:1) (Table 2). The yeast 20S proteasomes also failed to cleave the Q₂₀ (SEQ ID NO: 30) sequence in Q₂₀RRGRR (SEQ ID NO:7) but rapidly cleaved within the flanking RRGRR (SEQ ID NO:6). Without intending to be bound by theory, these data indicate that polyQ sequences, once within the eukaryotic proteasomes, are resistant to digestion and must exit as extended polyQ peptides, either intact or lacking the first glutamine residue (when it follows KK).

### EXAMPLE VI

### Prokaryotic Proteasomes Make Multiple Cuts Rapidly within the Q₂₀ Repeat (SEQ ID NO:30)

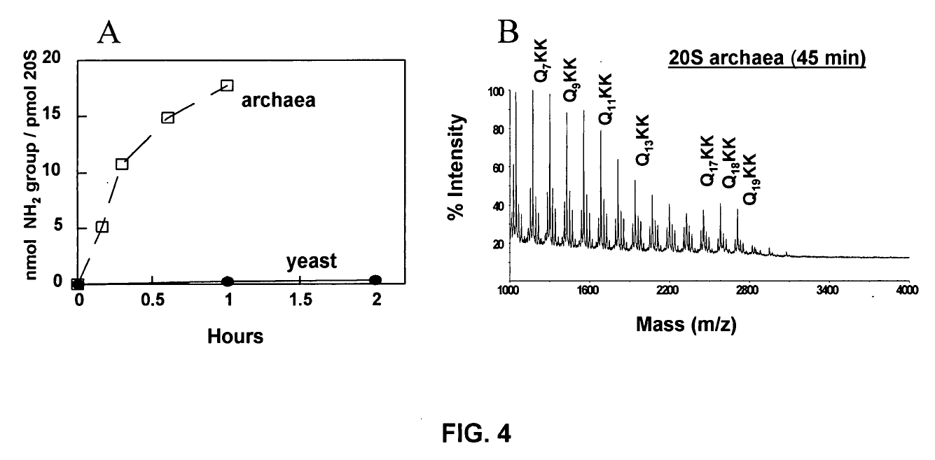

20S proteasomes of archaebacteria, e.g., Thermoplasma acidophilum, contain 14 identical active sites, which, though originally classified as “chymotrypsin-like” (Dahlmann et al. (1991) Biomed. Biochim. Acta 50:465, incorporated herein by reference in its entirety for all purposes), were later shown to cleave also after basic and acidic residues (Akopian et al., supra). These “less-specific” active sites were tested to determine if they could degrade within a polyQ sequence. A gated channel in the α-ring homologous to that in eukaryotic proteasomes regulates substrate entry into these particles. It was determined that mutant archaebacterial proteasomes lacking this gate (Δ7α) (Benaroudj et al., supra, incorporated herein by reference in its entirety for all purposes) cleaved polyQ peptides and control peptides at least 17 fold faster than the wild-type particles. Surprisingly, these archaebacterial ‘open-gate’ proteasomes cleaved both bKKQ₁₀KK (SEQ ID NO:8) and bKKQ₂₀KK (SEQ ID NO:9) at least 70 times faster than the yeast ‘open-gate’ 20S proteasomes (FIG. 4A) at identical incubation temperatures (37° C.). In fact, the archaebacterial proteasomes completely consumed the bKKQ₂₀KK (SEQ ID NO:9) within 45 minutes with half the concentration of the yeast particles, and the proteasome inhibitor MG132 inhibited this process. Therefore, the archaebacterial proteasomes, and not a contaminating peptidase, were responsible for rapid hydrolysis of bKKQ₂₀KK (SEQ ID NO:9). By contrast, yeast and archaebacterial particles hydrolyzed a control 13-residue peptide at similar rates. Thus, without intending to be bound by theory, the Thermoplasma proteasomes are far more active in degrading polyQ peptides than their eukaryotic counterparts.

When the products generated from bKKQ₂₀KK (SEQ ID NO:9) by the archaebacterial proteasomes were analyzed by MALDI-TOF, they were found to be very different from those generated by the eukaryotic particles. Q₁₉KK (SEQ ID NO:13) or the Q₁₈KK (SEQ ID NO:14) fragments were not the major products; instead, the archaebacterial proteasomes produced multiple short fragments with similar peak intensities ranging in size between Q₆KK (SEQ ID NO:31) and Q₁₂KK (SEQ ID NO:32) (FIG. 4B). Individual peaks differing in mass by one glutamine residue could be identified between Q₁₉KK (SEQ ID NO:13) and Q₆KK (SEQ ID NO:31), and the longer fragments were less abundant than shorter ones. Similarly, with bKKQ₁₀KK (SEQ ID NO:8) as the substrate, multiple cuts were made within the polyQ repeat, releasing Q₈KK (SEQ ID NO:18), Q₇KK (SEQ ID NO:33), and Q₆KK (SEQ ID NO:31). These findings demonstrated an ability of the archaebacterial proteasomes to cleave rapidly after a QQQ sequence. Without intending to be bound by theory, the failure of eukaryotic proteasomes to cut within the polyQ repeats is therefore due to their active site specificities and not to some inherent property of proteasome shape, its threonine-based catalytic mechanism, to aggregation of the polyQ peptides, or to the analytical approach.

### EXAMPLE VII

### Archaebacterial Proteasomes, Unlike Eukaryotic Proteasomes, can Digest a Q₃₅ Repeat (SEQ ID NO:34) While Degrading Q₃₅-Myoglobin (SEQ ID NO:35)

The findings with polyQ-peptides predicted that eukaryotic proteasomes, when degrading a polyQ protein, will release the non-degradable polyQ sequence intact. By contrast, the archaebacterial proteasomes should rapidly digest such repeats in proteins. To test these predictions, a protein fusion of sperm whale myoglobin with an extended Q₃₅ repeat (SEQ ID NO:34) taken from ataxin-3, the Machado-Joseph disease protein was used as a model protein substrate. This Q₃₅ repeat (SEQ ID NO:34) along with 15 flanking residues (LVYFEKQQQKQ₃₅RDLSLQ) (SEQ ID NO:36) was inserted between residues 45 and 48 of myoglobin. This fusion protein can be expressed in E. coli as a soluble holoprotein containing heme (Tanaka et al. (2001) J. Biol. Chem. 276:45470, incorporated herein by reference in its entirety for all purposes). The polyQ insert did not cause major perturbations of myoglobin structure but led to some destabilization of the surface of the protein.

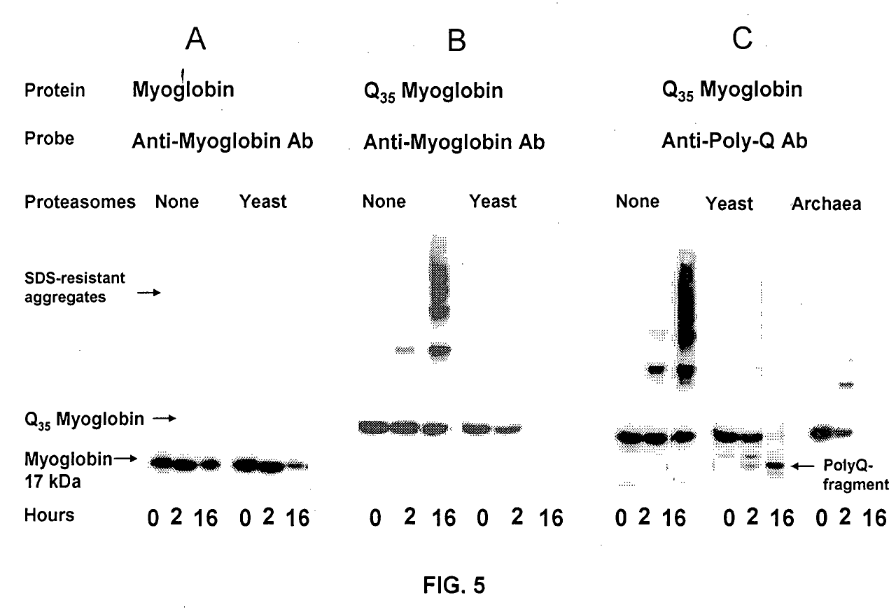

After extraction of the heme group, wild-type apomyoglobin was degraded by the 26S proteasomes, but the Q₃₅ apomyoglobin (SEQ ID NO:37) formed an insoluble aggregate with very little recovery of the soluble protein. However, the ‘open-gate’ forms of 20S yeast proteasomes and archaea were able to degrade wild-type myoglobin and the fusion protein at 37° C. These proteasomes are known to degrade unfolded proteins to small peptides (Benaroudj et al., supra; Kohler et al. (2001) Mol. Cell. 7:1143, incorporated herein by reference in its entirety for all purposes), but their ability to digest native myoglobin (FIG. 5A) or Q₃₅-myoglobin (SEQ ID NO:35) (FIG. 5B) at 37° C. in the absence of the ATP-dependent regulatory particles (19S or its archaebacterial homolog, PAN) came as a surprise.

To follow the fate of the myoglobin-derived sequences and the polyQ-repeat, an anti-myoglobin polyclonal antibody which recognized both the wild-type and the Q₃₅-myoglobin (SEQ ID NO:35) and a monoclonal anti-polyQ antibody (raised against the Q₃₈ repeats in the TATA Box binding protein) which did not react with wild-type myoglobin were used. In the absence of proteasomes, some of the Q35-myoglobin (SEQ ID NO:35) at 37° C. formed SDS resistant aggregates with time. After 2 hours of incubation, SDS-resistant dimers and tetramers were evident, and after 16 hours, higher molecular weight aggregates formed (broad smear in FIGS. 5B, C). The oligomers and aggregates reacted with both the anti-myoglobin (FIG. 5B) and anti-polyQ antibodies (FIG. 5C), but were not abundant enough to be detectable by Coomassie brilliant blue. When proteasomes were present, the Q₃₅-myoglobin (SEQ ID NO:35) was degraded, and no aggregates were detected. This fusion protein was degraded more rapidly than the wild-type, presumably because the repeat destabilizes the mutant protein.

During degradation of Q₃s-myoglobin (SEQ ID NO:35), no large polypeptide fragments could be detected on SDS-PAGE using the anti-myoglobin antibody (FIG. 5B). When the intact Q₃₅-myoglobin (SEQ ID NO:35) was almost completely digested, as shown with the anti-myoglobin antibody, the polyQ antibody still reacted with one major product, which migrated as a 17 kDa polypeptide (FIG. 5C). Thus, as predicted from the experiments with polyQ peptides, the polyQ repeat appeared to be released intact by the yeast proteasomes. An additional polyQ-containing band migrating at approximately 18 kDa was also generated from the Q₃₅-myoglobin (SEQ ID NO:35), but it was degraded further to the 17 kDa species with longer incubation times. The 17 kDa and 18 kDa fragments were not recognized by the polyclonal anti-myoglobin antibody and appeared to contain mainly the polyQ sequence. These findings confirm that these eukaryotic proteasomes cannot digest polyQ sequences in proteins, which therefore accumulate intact or with flanking sequences.

As predicted, the archaebacterial proteasomes differed markedly from the eukaryotic proteasomes in their capacity to handle the polyQ sequence in proteins. Unlike the yeast particles, those from the Thermoplasma, which readily hydrolyzed Q₁₀(SEQ ID NO:38) and Q₂₀ (SEQ ID NO:30) sequences in peptides, did not generate any large polyQ-containing fragments from Q₃₅-myoglobin (SEQ ID NO:35) (FIG. 5C), apparently because of their ability to digest aggregation-prone polyQ sequences.

### EXAMPLE VIII

### Discussion

The data presented herein demonstrate a new feature of proteasomal function that has important implications for understanding the pathway for degradation of polyQ-containing proteins and the pathogenesis of polyQ-diseases and for treating bacterial infections. Using synthetic peptides containing 10-30 Q residues, it was determined that mammalian and yeast proteasomes cannot digest long-glutamine repeats. The only cleavages made in these peptides were rapid cuts within the flanking basic residues in Q₂₀RRGRR (SEQ ID NO:7) and slow cleavages after bKKQ in bKKQ₁₀KK (SEQ ID NO:8) and bKKQ₂₀KK (SEQ ID NO:9) or after GKKQ (SEQ ID NO:39) in GAPVPYPDPLEPRGG-KKQ₂₀KK (SEQ ID NO:40). Also, increasing the length of the Q-repeat in bKKQ_{N}KK (SEQ ID NO:41) from 10 to 30, which should increase the target size of any enzyme that can cut within a polyQ repeat, failed to increase the rate of cleavage. Since the cleavages occurred in the flanking sequence or after the initial Q, these substrates clearly were able to enter the particles. Surprisingly, the flanking regions, though included only to maintain the polyQ sequence soluble, actually determined the site and rate of cleavages. This failure to digest the polyQ chains was observed with all forms of eukaryotic proteasomes tested, including mammalian 26S complexes and 20S particles activated either by 0.02% SDS or the PA28αβ complex, and the yeast 20S α3ΔN mutant. In all these forms, the substrate entry channel is in an open state, which was necessary to obtain sufficient products for mass spectrometry.

The inability of eukaryotic proteasomes to degrade a polyQ sequence was confirmed with a Q₃₅-myoglobin (SEQ ID NO:35) fusion whose polyQ-repeat and flanking residues originated from ataxin-3, (Machado-Joseph protein) (Tanaka et al. supra, incorporated herein by reference in its entirety for all purposes). While all the myoglobin-derived sequences recognized by the anti-myoglobin polyclonal antibody were degraded, the polyQ repeat remained intact. Without intending to be bound by theory, like most substrates, polyQ proteins are probably ubiquitinated before being targeted to the 19S complex, where they undergo ATP-dependent unfolding and translocation into the 20S particle (Ciechanover et al. (2003) Neuron 40:427, incorporated herein by reference in its entirety for all purposes). By using open-gate forms of 20S proteasomes with the Q₃₅-myoglobin fusion, these reactions could be bypassed in order to focus on the final degradative process.

In contrast to eukaryotic proteasomes, the ancestral particles from the archaebacterium T. acidophilum digested polyQ peptides very rapidly and degraded completely the myoglobin-polyQ fusion protein. Unlike the eukaryotic homologs, these prokaryotic proteasomes contain 14 identical active sites of broad specificity. Their ability to rapidly digest polyQ sequences indicates that their active sites can bind a row of three Q-residues, which the specialized active sites in eukaryotic proteasomes cannot do. The data presented herein indicate that the trypsin-like sites can cleave after a P1 glutamine if the preceding residue at P2 is a lysine and at P3 a αN-blocked lysine. In related experiments, it was confirmed that the active sites of eukaryotic proteasomes have a low affinity for polyQ sequences, since bKKQ₁₀KK (SEQ ID NO:8) could not competitively inhibit the hydrolysis of standard fluorogenic substrates. Thus, their failure to degrade polyQ-sequences is due to the inability of their active sites to bind the polyQ repeat, not to the proteasome's unique catalytic mechanism, which is similar in eukaryotic and archaebacterial particles.

### EXAMPLE IX

### Experimental Procedures

All polyQ peptides were prepared and solubilized as described previously (Chen et al. (2001) Protein Sci. 10:887, incorporated herein by reference in its entirety for all purposes) and stored frozen at −80° C. Just before use, peptides were thawed, centrifuged, and neutralized to pH 7.5 in assay buffers. Amino acid compositions of bKKQ₁₀₋₃₀KK (SEQ ID NO:41) peptides were verified by amino acid analysis. The molecular weights of all peptides were determined by MALDI-TOF and corresponded to their theoretical molecular weights, (except for Q₂₀RRGRR (SEQ ID NO:7); see Table 2) and appeared to be >95% pure by amino acid analysis. However, substrates with 20 and 30Qs were found not to be pure and contained contaminant peptides, which lacked one or two glutamine residues that were detected by MALDI-TOF but not revealed by amino acid analysis. Such contaminants presumably resulted from occasional inefficiency in the coupling of glutamine residue to the growing peptide chain (Chen et al. (2002) Biochemistry 41:7391, incorporated herein by reference in its entirety for all purposes) and which could not be removed by HPLC.

QATVGDINTERPGMLDFTGK (SEQ ID NO:12), a peptide fragment from the diazepam binding inhibitory protein was obtained from CALBIOCHEM® (EMD Biosciences, San Diego, Calif.). Peptide concentrations were determined by UV absorbance at 196 nm (Mayer and Miller (1970) Anal. Biochem. 36:91, incorporated herein by reference in its entirety for all purposes).

Rabbit 20S and 26S proteasomes were purified as described previously with some variations (Kisselev et al. (2002) supra, incorporated herein by reference in its entirety for all purposes). Yeast expressing the ‘open-gate’ 20S proteasomes (α3ΔN) mutant was a kind gift from Dr. Daniel Finley. These particles, which were active in the absence of SDS normally used to stimulate the wild-type 20S proteasomes, were purified as mentioned above, but the buffer did not contain ATP.

For 20S proteasomes, 20 mM Hepes (pH 7.5) containing 1 mM dithiothreitol (DTT) was used. For reactions with 26S proteasomes, the buffer also contained 0.5 mM ATP and 2.5 mM MgCl₂. All reactions were performed at 37° C. 5 μl aliquots were withdrawn at different times, 50 μl of 0.2M sodium phosphate (pH 6.8) was added and then mixed vigorously with 50 μl fluorescamine for 3 minutes. The reaction was terminated with 0.4 ml of ice-cold water and fluorescence read at 380 nm excitation and 470 nm emission.

For studies with PA 28αβ, 350 ng of rabbit 20S proteasomes were incubated with or without 5 μg of bovine PA 28αβ (Boston Biochem, Inc., Cambridge, Mass.) for 10 minutes in a total volume of 10 μl. 80 μM bKKQ₁₀KK (SEQ ID NO:8) was subsequently added to a total volume to 40 μl. 0.8 μg rabbit 20S proteasomes were incubated with 80 μM of bKKQ₁₀KK (SEQ ID NO:8) in 20S buffer containing 0.02% SDS in a total volume of 50 μl.

Product Analysis by MALDI-TOF

Aliquots of the reaction mixture were directly analysed by Matrix-assisted laser desorption/ionization-time of flight mass spectrometry (MALDI-TOF MS) (Applied Biosystems Voyager 4036, at the Dana-Farber Core Facility). Reflector mode of analysis was performed on reaction mixtures with bKKQ₁₀KK (SEQ ID NO:8) peptides, and for reactions with the Q₂₀ peptides (SEQ ID NO:30), linear mode of analysis was used to increase the sensitivity of detection due to poor ionization of these peptides with long glutamine sequences.

Reactions of Q₂₀RRGRR (SEQ ID NO:7) with mammalian 26S proteasomes, after 10 hours incubation, were analyzed for product formation. Concentrations are as given under fluorescamine assay. Reactions with yeast ‘open-gate’ mutant contained 3.5 μg proteasomes in a total volume of 50 μl. For reactions with bKKQ₂₀KK (SEQ ID NO:9), the peptide, (35 μM) was incubated either alone or with rabbit 26S or 3.5 μg of yeast ‘open-gate’ 20S proteasomes in a total volume of 40 μl for 10 hours.

Degradation of Myoglobin and O₃₅-Myoglobin by Proteasomes

Recombinant sperm whale Myoglobin and Q₃₅-myoglobin (SEQ ID NO:35) were expressed and purified as described previously (Tanaka et al. supra) and stored at −80° C. 10 μM of myoglobin or Q₃₅-myoglobin (SEQ ID NO:35) was incubated with yeast (37 pmol) and archaebacterial (14 pmol) ‘open-gate’ proteasomes in 20S buffer, and aliquots (10 μl) were removed at different times, separated on a 4-12% Bis-Tris SDS gel using 2-(N-morpholino) ethane sulfonic acid (MES) buffer (Invitrogen, Carlsbad, Calif.), transferred to PVDF membrane (HYBOND™ P, Amersham Biosciences, Piscataway, N.J.), and probed with anti-myoglobin antibody coupled to HRP (Bethyl Laboratories Inc., Montgomery, Tex.). For Q₃₅-myoglobin (SEQ ID NO:35), the same blot was subsequently probed with a mouse anti-polyQ monoclonal antibody, which was raised originally against TATA-Box protein with 38 glutamine residues in a row (catalog number MAB1574, Chemicon, Temecula, Calif.) followed by anti-mouse alkaline phosphatase.

### EXAMPLE X

### References

Each reference is incorporated herein by reference in its entirety for all purposes.
Altschuler et al. (1997) J. Pept. Res. 50:73
Bence et al. (2001) Science 292:1552
Berke et al. (2003) Curr. Opin. Genet. Dev. 13:253
Chai et al. (2001) J. Biol. Chem. 276:44889
Chung et al. (2001) Trends Neurosci. 24:S7
Davies et al. (1997). Cell 90:537
DiFiglia et al. (1997) Science 277:1990
Dyer et al. (2001) Nat. Genet. 29:270
Goellner et al. (2003) Int. J. Biochem. Cell Biol. 35:562
Jana et al. (2001). Hum. Mol. Genet. 10: 1049
Kazantsev et al. (1999) Proc. Natl. Acad. Sci. USA 96:11404
Li et al. (1998) Hum. Mol. Genet. 7:777
Sakahira et al. (2002) Proc. Natl. Acad. Sci. USA 99 Suppl. 4:16412
Sanchez et al. (2003) Nature 421:373
Schmidt et al. (2002) Ann. Neurol. 51:302
Steffan et al. (2000) Proc. Natl. Acad. Sci. USA 97:6763
Verhoef et al. (2002) Hum. Mol. Genet. 11:2689
Waelter et al. (2001) Mol. Biol. Cell 12:1393
Yang et al. (2002) Hum. Mol. Genet. 11:2905
Zoghbi et al. (2000) Ann. Rev. Neurosci. 23:217

What is claimed is:

1. A method for therapeutically treating a bacterial infection in a human or non-human mammal in need thereof, the method comprising administering to the human or non-human mammal a glutamine-glutamine dipeptide, a glutamine-glutamine-glutamine tripeptide or a polypeptide comprising a polyQ domain, and a pharmaceutically acceptable carrier.

2. The method of claim 1, wherein the dipeptide, tripeptide or polypeptide comprises an amino-terminal blocking moiety.

3. The method of claim 2, wherein the amino-terminal blocking moiety is selected from the group consisting of: N-acetyl, N-formyl, tert-butylcarbonyl and para-nitrophenylfornate.

4. The method of claim 1, wherein the dipeptide, tripeptide or polypeptide comprises a carboxy-terminal group that reacts with an active site of a proteasome.

5. The method of claim 4, wherein the carboxy-terminal group is selected from the group consisting of: boronic acid, aldehyde, vinyl sulfone, epoxyketone and beta lactone ring.

6. The method of claim 1, wherein an activity of a bacterial proteasome is inhibited.

7. The method of claim 1, wherein the bacterial infection is a Mycobacterium tuberculosis infection.

8. A method for therapeutically treating one or more symptoms associated with a bacterial infection in a human or non-human mammal in need thereof, the method comprising administering to the human or the non-human mammal a glutamine-glutamine dipeptide, a glutamine-glutamine-glutamine tripeptide or a polypeptide comprising a polyQ domain, and a pharmaceutically acceptable carrier.

9. The method of claim 8, wherein the dipeptide, tripeptide or polypeptide comprises an amino-terminal blocking moiety.

10. The method of claim 9, wherein the amino-terminal blocking moiety is selected from the group consisting of: N-acetyl, N-formyl, tert-butylcarbonyl and para-nitrophenylfornate.

11. The method of claim 8, wherein the dipeptide, tripeptide or polypeptide comprises a carboxy-terminal group that reacts with an active site of a proteasome.

12. The method of claim 11, wherein the carboxy-terminal group is selected from the group consisting of: boronic acid, aldehyde, vinyl sulfone, epoxyketone and beta lactone ring.

13. The method of claim 8, wherein the symptoms are selected from the group consisting of: chest pain, non-productive coughing, coughing up blood, coughing up sputum, weakness, fatigue, weight loss, loss of appetite, chills, fever and night sweats.

14. The method of claim 8, wherein an activity of a bacterial proteasome is inhibited.

15. The method of claim 8, wherein the bacterial infection is a Mycobacterium tuberculosis infection.

16. A method for killing a cell infected with a bacterium, said method comprising:
contacting the cell with a glutamine-glutamine dipeptide, a glutamine-glutamine-glutamine tripeptide or a polypeptide comprising a polyQ domain;
inhibiting an activity of a bacterial proteasome; and
allowing the cell infected with a bacterium to be killed.

17. The method of claim 16, wherein the cell infected with a bacterium is a macrophage.

18. A method for killing a bacterial cell, said method comprising:
contacting the cell with a glutamine-glutamine dipeptide, a glutamine-glutamine-glutamine tripeptide or a polypeptide comprising a polyQ domain;
inhibiting an activity of a bacterial proteasome; and
allowing the bacterial cell to be killed.

19. The method of claim 18, wherein the bacterial cell is present within a macrophage phagosome.

20. A method for therapeutically treating a polyglutamine disorder in a human or non-human mammal in need thereof, the method comprising administering to the human or non-human mammal a prokaryotic 20S proteasome or a portion thereof, and a pharmaceutically acceptable carrier.

21. The method of claim 20, wherein the polyglutamine disorder is neurodegenerative disorder.

22. The method of claim 20, wherein the polyglutamine disorder is selected from the group consisting of: Huntington's disease, spinocerebellar ataxia type 1, spinocerebellar ataxia type 2, spinocerebellar ataxia type 3, spinocerebellar ataxia type 6, spinocerebellar ataxia type 7, dentatorubral-pallidoluysian atrophy, spinobulbar muscular atrophy, oculopharyngeal muscular dystrophy, and Huntington's disease-like Type 2.

23. A pharmaceutical composition for therapeutically treating a bacterial infection, the pharmaceutical composition comprising a glutamine-glutamine dipeptide, a glutamine-glutamine-glutamine tripeptide or a polypeptide comprising a polyQ domain, and a pharmaceutically acceptable carrier.

24. The pharmaceutical composition of claim 23, wherein the dipeptide, tripeptide or polypeptide comprises an amino-terminal blocking moiety.

25. The pharmaceutical composition of claim 24, wherein the amino-terminal blocking moiety is selected from the group consisting of: N-acetyl, N-formyl, tert-butylcarbonyl and para-nitrophenylfornate.

26. The pharmaceutical composition of claim 23, wherein the dipeptide, tripeptide or polypeptide comprises a carboxy-terminal group that reacts with an active site of a proteasome.

27. The pharmaceutical composition of claim 26, wherein the carboxy-terminal group is selected from the group consisting of: boronic acid, aldehyde, vinyl sulfone, epoxyketone and beta lactone ring.

28. A pharmaceutical composition for therapeutically treating one or more symptoms associated with a bacterial infection, the pharmaceutical composition comprising a glutamine-glutamine dipeptide, a glutamine-glutamine-glutamine tripeptide or a polypeptide comprising a polyQ domain, and a pharmaceutically acceptable carrier.

29. The pharmaceutical composition of claim 28, wherein the dipeptide, tripeptide or polypeptide comprises an amino-terminal blocking moiety.

30. The pharmaceutical composition of claim 29, wherein the amino-terminal blocking moiety is selected from the group consisting of: N-acetyl, N-formyl, tert-butylcarbonyl and para-nitrophenylfornate.

31. The pharmaceutical composition of claim 28, wherein the dipeptide, tripeptide or polypeptide comprises a carboxy-terminal group that reacts with an active site of a proteasome.

32. The pharmaceutical composition of claim 31, wherein the carboxy-terminal group is selected from the group consisting of: boronic acid, aldehyde, vinyl sulfone, epoxyketone and beta lactone ring.

33. The pharmaceutical composition of claim 28, wherein the symptoms are selected from the group consisting of: chest pain, non-productive coughing, coughing up blood, coughing up sputum, weakness, fatigue, weight loss, loss of appetite, chills, fever and night sweats.


SEQUENCE LISTING (40 sequences)

SEQ ID NO: 1 - protein, 23 aa, Artificial Sequence
  Peptide generated by solid-phase peptide synthesis
  MISC_FEATURE (1)..(1): biotin attached to alpha amino group
KKQQQQQQQQQQQQQQQQQQQKK

SEQ ID NO: 2 - protein, 22 aa, Artificial Sequence
  Peptide generated by solid-phase peptide synthesis
  MISC_FEATURE (1)..(1): Biotin attached to alpha amino group
KKQQQQQQQQQQQQQQQQQQKK

SEQ ID NO: 3 - protein, 18 aa, Artificial Sequence
  Peptide generated by solid-phase peptide synthesis
GAPVPYPDPLEPRGGKKQ

SEQ ID NO: 4 - protein, 24 aa, Artificial Sequence
  Peptide generated by solid-phase peptide synthesis
QQQQQQQQQQQQQQQQQQQRRGRR

SEQ ID NO: 5 - protein, 14 aa, Artificial Sequence
  Peptide generated by solid-phase peptide synthesis
  MISC_FEATURE (1)..(1): Biotin attached to alpha amino group
KKQQQQQQQQQQKK

SEQ ID NO: 6 - protein, 5 aa, Artificial Sequence
  Peptide generated by solid-phase peptide synthesis
RRGRR

SEQ ID NO: 7 - protein, 25 aa, Artificial Sequence
  Peptide generated by solid-phase peptide synthesis
QQQQQQQQQQQQQQQQQQQQRRGRR

SEQ ID NO: 8 - protein, 14 aa, Artificial Sequence
  Peptide generated by solid-phase peptide synthesis
  MISC_FEATURE (1)..(1): Biotin attached to alpha amino group
KKQQQQQQQQQQKK

SEQ ID NO: 9 - protein, 24 aa, Artificial Sequence
  Peptide generated by solid-phase peptide synthesis
  MISC_FEATURE (1)..(1): Biotin attached to alpha amino group
KKQQQQQQQQQQQQQQQQQQQQKK

SEQ ID NO: 10 - protein, 24 aa, Artificial Sequence
  Peptide generated by solid-phase peptide synthesis
KKQQQQQQQQQQQQQQQQQQQQKK

SEQ ID NO: 11 - protein, 22 aa, Artificial Sequence
  Peptide generated by solid-phase peptide synthesis
QQQQQQQQQQQQQQQQQQQRRG

SEQ ID NO: 12 - protein, 20 aa, Artificial Sequence
  Peptide generated by solid-phase peptide synthesis
QATVGDINTERPGMLDFTGK

SEQ ID NO: 13 - protein, 21 aa, Artificial Sequence
  Peptide generated by solid-phase peptide synthesis
QQQQQQQQQQQQQQQQQQQKK

SEQ ID NO: 14 - protein, 20 aa, Artificial Sequence
  Peptide generated by solid-phase peptide synthesis
QQQQQQQQQQQQQQQQQQKK

SEQ ID NO: 15 - protein, 19 aa, Artificial Sequence
  Peptide generated by solid-phase peptide synthesis
QQQQQQQQQQQQQQQQQKK

SEQ ID NO: 16 - protein, 4 aa, Artificial Sequence
  Peptide generated by solid-phase peptide synthesis
  MISC_FEATURE (1)..(1): N-terminal succinyl
  MISC_FEATURE (4)..(4): C-terminal 7-methoxycoumarin
LLVY

SEQ ID NO: 17 - protein, 11 aa, Artificial Sequence
  Peptide generated by solid-phase peptide synthesis
QQQQQQQQQKK

SEQ ID NO: 18 - protein, 10 aa, Artificial Sequence
  Peptide generated by solid-phase peptide synthesis
QQQQQQQQKK

SEQ ID NO: 19 - protein, 13 aa, Artificial Sequence
  Peptide generated by solid-phase peptide synthesis
  MISC_FEATURE (1)..(1): Biotin attached to alpha amino group
KKQQQQQQQQQKK

SEQ ID NO: 20 - protein, 4 aa, Artificial Sequence
  Peptide generated by solid-phase peptide synthesis
  MOD_RES (1)..(1): ACETYLATION
  MISC_FEATURE (4)..(4): C-terminal vinyl sulfone
YRLN

SEQ ID NO: 21 - protein, 15 aa, Artificial Sequence
  Peptide generated by solid-phase peptide synthesis
GAPVPYPDPLEPRGG

SEQ ID NO: 22 - protein, 20 aa, Artificial Sequence
  Peptide generated by solid-phase peptide synthesis
QATVGDINTERPGMLDFTGK

SEQ ID NO: 23 - protein, 34 aa, Artificial Sequence
  Peptide generated by solid-phase peptide synthesis
  MISC_FEATURE (1)..(1): Biotin attached to alpha amino group
KKQQQQQQQQQQQQQQQQQQQQQQQQQQQQQQKK

SEQ ID NO: 24 - protein, 22 aa, Artificial Sequence
  Peptide generated by solid-phase peptide synthesis
QQQQQQQQQQQQQQQQQQQQKK

SEQ ID NO: 25 - protein, 23 aa, Artificial Sequence
  Peptide generated by solid-phase peptide synthesis
QQQQQQQQQQQQQQQQQQQQRRG

SEQ ID NO: 26 - protein, 22 aa, Artificial Sequence
  Peptide generated by solid-phase peptide synthesis
QQQQQQQQQQQQQQQQQQQQRR

SEQ ID NO: 27 - protein, 21 aa, Artificial Sequence
  Peptide generated by solid-phase peptide synthesis
QQQQQQQQQQQQQQQQQQQRR

SEQ ID NO: 28 - protein, 20 aa, Artificial Sequence
  Peptide generated by solid-phase peptide synthesis
QQQQQQQQQQQQQQQQQQQR

SEQ ID NO: 29 - protein, 13 aa, Artificial Sequence
  Peptide generated by solid-phase peptide synthesis
YGGFLRRIRPKLK

SEQ ID NO: 30 - protein, 20 aa, Artificial Sequence
  Peptide generated by solid-phase peptide synthesis
QQQQQQQQQQQQQQQQQQQQ

SEQ ID NO: 31 - protein, 8 aa, Artificial Sequence
  Peptide generated by solid-phase peptide synthesis
QQQQQQKK

SEQ ID NO: 32 - protein, 14 aa, Artificial Sequence
  Peptide generated by solid-phase peptide synthesis
QQQQQQQQQQQQKK

SEQ ID NO: 33 - protein, 9 aa, Artificial Sequence
  Peptide generated by solid-phase peptide synthesis
QQQQQQQKK

SEQ ID NO: 34 - protein, 35 aa, Artificial Sequence
  Peptide generated by solid-phase peptide synthesis
QQQQQQQQQQQQQQQQQQQQQQQQQQQQQQQQQQQ

SEQ ID NO: 35 - protein, 35 aa, Artificial Sequence; identical to SEQ ID NO: 34
  Peptide generated by solid-phase peptide synthesis
  MISC_FEATURE (35)..(35): C-terminus linked to myoglobin

SEQ ID NO: 36 - protein, 51 aa, Artificial Sequence
  Peptide generated by solid-phase peptide synthesis
LVYFEKQQQKQQQQQQQQQQQQQQQQQQQQQQQQQQQQQQQQQQQRDLSLQ

SEQ ID NO: 37 - protein, 35 aa, Artificial Sequence; identical to SEQ ID NO: 34
  Peptide generated by solid-phase peptide synthesis
  MISC_FEATURE (35)..(35): C-terminus linked to apomyoglobin

SEQ ID NO: 38 - protein, 10 aa, Artificial Sequence
  Peptide generated by solid-phase peptide synthesis
QQQQQQQQQQ

SEQ ID NO: 39 - protein, 4 aa, Artificial Sequence
  Peptide generated by solid-phase peptide synthesis
GKKQ

SEQ ID NO: 40 - protein, 39 aa, Artificial Sequence
  Peptide generated by solid-phase peptide synthesis
GAPVPYPDPLEPRGGKKQQQQQQQQQQQQQQQQQQQQKK